(19) United States
(12) Patent Application Publication
(10) Pub. No.: US 2019/0298187 A1
(43) Pub. Date: Oct. 3, 2019

(54) FLOW SENSOR AND METHOD OF MEASURING A FLOW RATE

(71) Applicant: KONINKLIJKE PHILIPS N.V., EINDHOVEN (NL)

(72) Inventors: CORNELIS PETRUS HENDRIKS, EINDHOVEN (NL); DAAN ANTON VAN DEN ENDE, BREDA (NL); RONALD ANTONIE HOVENKAMP, EINDHOVEN (NL); MARK THOMAS JOHNSON, ARENDONK (BE); ARJEN VAN DER HORST, TILBURG (NL); ACHIM HILGERS, ALSDORF (DE)

(21) Appl. No.: 16/462,609

(22) PCT Filed: Nov. 28, 2017
(86) PCT No.: PCT/EP2017/080643

§ 371 Date: May 21, 2019

(30) Foreign Application Priority Data
Nov. 28, 2016 (EP) 16200841.1

(51) Int. Cl. A61B 5/0265; G01F 1/688; G01F 1/66; A61B 5/00; G01F 15/02

(52) U.S. Cl. CPC A61B 5/0265; G01F 1/6882; A61B 2562/0247; A61B 5/6852; G01F 15/022; G01F 1/66

(57) ABSTRACT

A flow sensor comprises an electroactive material device. A driver controls the electroactive material device to deliver heat locally to the flowing medium for which the flow is to be sensed. Temperature sensing signals are obtained and these are used to derive a flow measurement. The way the heat is dissipated relates to the flow, and it is measurable based on the temperature sensing signals. The temperature sensing involves measuring an electrical characteristic which comprises an impedance or an impedance phase angle of the electroactive material device at at least a first frequency and at a second frequency different from the first frequency. The influences of temperature and pressure can in this way be decoupled so that the temperature can be measured at any pressure.

### FIELD OF THE INVENTION

This invention relates to flow sensors and in particular for measuring blood flow.

### BACKGROUND OF THE INVENTION

Blood flow measurement is of interest for many different diagnostic reasons. One example is for diagnosis of stenosis, which is a form of arterial disease wherein the blood flow is restricted due to a local narrowing of the blood vessel, e.g. due to plaque formed at the vessel wall.

Stenosis evaluation and treatment can be supported with guidewire sensors (CMUT, piezo-crystal, resistors) which either measure local blood flow, or local blood pressure. However, the complex hemodynamics of stenosis is not sufficiently explained by either pressure or flow alone. Therefore wires are being developed with multiple sensors but this leads to complex devices.

Furthermore, the flow sensors are complicated devices and a simpler sensing approach would be of interest.

In addition to a sensing function, the guidewires also preferably have good steerability in small and tortuous vessels. Integrating a mechanical actuator for tip steering may be used to implement steerability, but on the other hand it increases the device complexity.

WO 2006/135293 discloses an implantable flow sensor based on heating and subsequent analysis of the cooling caused by the flow. U.S. Pat. No. 4,726,225 discloses a flow rate meter which measures flow based on a temperature decrease.

It would therefore be desirable to have a simple sensor design able to measure flow, and preferably also which could form part of a single multifunction component for flow sensing as well as pressure sensing and/or actuation.

### SUMMARY OF THE INVENTION

According to examples in accordance with an aspect of the invention, there is provided a flow sensor comprising:

an electroactive material device arrangement;

a driver for controlling the electroactive material device arrangement to deliver heat locally to the flowing medium for which the flow is to be sensed; and

a controller adapted to:
read sensing signals from the electroactive material device arrangement, which sensing signals relate to the temperature at the electroactive material device; and
use the sensing signals to derive a flow measurement,

wherein the controller is adapted to read sensing signals by providing sensor readings for performing measurements of an electrical characteristic which comprises an impedance or an impedance phase angle of the electroactive material device at at least a first frequency and at a second frequency different from the first frequency, wherein the controller is adapted to derive from the measurements a temperature at the electroactive material sensor.

This arrangement uses a electroactive material device arrangement (where the “arrangement” may have one or more individual electroactive material devices) to deliver heat to a medium and then to monitor or control the resulting temperature such that the flow conditions (which take heat away) can be determined. The cooling rate may be monitored or the electrical heating may be monitored that is required to maintain a particular temperature.

The device may be able to measure pressure (or force) and temperature, even during actuation. This can be achieved by using the superposition of a drive signal and the measurement signals. The use of a small amplitude, high frequency electrical signal measured alternatingly at two different frequencies enables the influences of temperature and pressure to be decoupled. In this way, the temperature can be measured at any pressure. Furthermore, the pressure level may also be obtained if desired.

The driver may be adapted to provide drive signals at a frequency above a resonance frequency of the electroactive material device. This means the drive signals deliberately result in local heating, and are thus not the most efficient signals for actuation of the electroactive material.

In one arrangement, the driver is adapted to deliver heat during a predetermined time period, and the controller is adapted to read the sensing signals to monitor a subsequent temperature decay function, and thereby convert the evolution of the sensing signals over time to a flow measurement.

The way heat is taken away from the area to be sensed is thus monitored.

The controller may for example be adapted to measure a time period until the temperature reaches a reference temperature, and thereby convert the evolution of the sensing signals over time to a flow measurement.

In another arrangement, the driver is adapted to deliver heat continuously during a flow sensing time period, and the controller is adapted to read the sensing signals to monitor a steady state temperature. A steady state temperature is in this way monitored in response to a known heat delivery.

In yet another arrangement, the driver is adapted to deliver heat during a flow sensing time period, and the controller is adapted to control the heat delivery rate to achieve a predetermined steady state temperature. A heat delivery amount is in this way monitored in order to achieve a known temperature. For this purpose, the controller may control a duty cycle or frequency of heat delivery pulses.

In all examples above, the electroactive material device arrangement may comprise a single electroactive material device, functioning both as a heater and as a temperature sensor.

Instead, the electroactive material device arrangement may comprise an arrangement of a first electroactive material device functioning as a heater and second and third electroactive material devices functioning as sensors. They may be on opposite sides of the heater, so that heat gradients on each side of the heater can be monitored.

The electroactive material device arrangement may further function as a pressure sensor and/or an actuator. Thus, the same device may be used for flow measurement, for actuation (such as steering of a probe) and/or for pressure sensing. The pressure sensing may for example be used for load pressure sensing, for example against the skin.

The pressure sensor may be used to measure an external force or pressure (external means on the outer surface of the EAP). The external force or pressure can result from on-body skin contact, or from in-body blood vessel wall contact, or from in-body blood pressure in an artery.

The quantitative relation between blood pressure and the response of a particular EAP actuator configuration will be calibrated as part of the product development.

The invention makes used of measurements at two frequencies.

The first frequency is for example a resonance frequency at which the impedance or impedance phase angle has a maximum or minimum value, such as an anti-resonance frequency. The measurement at this frequency may be used to determine an external force or pressure.

When a signal is applied at a frequency matching the (undamped) anti-resonance frequency, a sudden mismatch induced by the applied load is for example detected as a consequent drop in impedance as measured across the sensor.

It is alternatively possible to use a driving signal which matches the (undamped) resonance frequency. In this case, the mismatch may be detected as a consequent jump in impedance measured across the sensor. In either case, the high frequency signal, in this way, allows for sensing of external pressure or force applied to the device at the same time as actuation.

The second frequency may be a frequency at which the electrical characteristic is constant with respect to load. Instead, it has a variation with temperature, and can thus be used for temperature measurement.

The control system may be adapted to apply a drive signal onto which measurement signals of the first and second frequencies are superposed, wherein the drive signal comprises a DC drive level or an AC drive signal with a frequency below the first and second frequencies.

By superposing a low-amplitude, high frequency sensing signal on top of a higher amplitude primary actuation signal, sensing and actuation functions may be achieved simultaneously.

The two, different frequency, measurement signals may be applied in sequence. Alternatively the different frequency measurements may be superimposed, since the size of the off-resonance frequency can be freely chosen.

The invention will work with electroactive materials in general. However, particularly useful materials are organic electroactive materials and/or polymeric electroactive materials. These have the electroactive characteristics, a suitable temperature dependence and also have ease of processing for them to be integrated in devices such as in body lumen (e.g. catheters). The electroactive material (polymer) may comprise a relaxor ferroelectric. By way of non-limiting example of such polymeric materials, ter-polymers (i.e. PVDF-TrFE-CFE or PVDF-TrFE-CTFE) relaxor ferroelectrics may be used. They are non-ferroelectric in the absence of an applied field, meaning that there is no electromechanical coupling when no drive signal is applied. When a DC bias signal is applied, for example, the electromagnetic coupling becomes non-zero. Relaxor ferroelectrics provide larger magnitudes of actuation deformation, and greater sensing sensitivity compared with other known EAP materials.

However, the device is not limited to the use of relaxor ferroelectrics, and piezoelectric EAP materials (such as, by way of example only, PVDF or PVDF-TrFE), may also for example be used in embodiments.

The sensor may form part of a catheter or guidewire.

Examples in accordance with a second aspect of the invention provide a method of measuring a flow rate comprising:

controlling an electroactive material device arrangement to deliver heat locally to the flowing medium for which the flow rate is to be measured;

reading sensing signals from the electroactive material device arrangement, which sensing signals relate to the temperature at the electroactive material device; and

using the sensing signals to derive a flow measurement,

wherein reading sensing signals comprises providing sensor readings for performing measurements of an electrical characteristic which comprises an impedance or an impedance phase angle of the electroactive material device at at least a first frequency and at a second frequency different from the first frequency, and deriving from the measurements a temperature at the electroactive material sensor.

The method may comprise providing drive signals at a frequency above a resonance frequency of the electroactive material device.

In one approach, the method comprises delivering heat during a predetermined time period, and reading the sensing signals to monitor a subsequent temperature decay function, and thereby converting the evolution of the sensing signals over time to a flow measurement. A time period may for example be measured until the temperature reaches a reference temperature, and thereby converting the evolution of the sensing signals over time to a flow measurement.

In another approach, the method comprises delivering heat continuously during a flow sensing time period, and reading the sensing signals to monitor a steady state temperature.

In another approach, the method comprises delivering heat during a flow sensing time period, and controlling the heat delivery rate to achieve a predetermined steady state temperature.

The method may additionally comprise pressure sensing and/or actuation.

### BRIEF DESCRIPTION OF THE DRAWINGS

Examples of the invention will now be described in detail with reference to the accompanying drawings, in which:

FIG. 1 shows a known electroactive polymer device which is not clamped;

FIG. 2 shows a known electroactive polymer device which is constrained by a backing layer;

FIG. 3 shows a flow sensor device;

FIG. 4 shows a first way to measure flow rate in dependence on a temperature function;

FIG. 5 shows a second way to measure flow rate based on a temperature function;

FIG. 6 shows a third way to measure flow rate based on a temperature function;

FIG. 7 shows a fourth way to measure flow rate based on a temperature function;

FIG. 8 shows a flow sensor device installed at the tip of a catheter;

FIG. 9 shows a first example of electroactive polymer device to explain the temperature measurement method;

FIG. 10 shows a calibration method;

FIG. 11 is a graph to show how a sensor only function may be used;

FIG. 12 shows a sensing method for use after the calibration;

FIG. 13 shows the electroactive polymer device of FIG. 3 in more detail;

FIG. 14 shows one equivalent circuit of an EAP device;

FIG. 15 shows changes in resistance and capacitance with frequency;

FIG. 16 shows changes in with frequency for two different actuation voltages;

FIG. 17 shows how the difference between the plots of FIG. 10 can be used to identify resonance frequencies;

FIG. 18 shows the dependency the impedance on the load for different temperatures at resonance;

FIG. 19 shows the dependency the impedance on the load for different temperatures away from resonance;

FIG. 20 shows the reproducibility of the temperature-impedance function;

FIG. 21 shows how temperature compensation may be used to improve load sensing;

FIG. 22 is used to explain how phase measurements may be used.

FIG. 23 shows the sensitivity of an example material with a certain composition versus temperature;

FIG. 24 shows the relationship between sensitivity and composition;

FIG. 25 shows first measurement results to demonstrate the feasibility of the heating function;

FIG. 26 shows second measurement results to demonstrate the feasibility of the heating function; and

FIG. 27 shows third measurement results to demonstrate the feasibility of the heating function.

### DETAILED DESCRIPTION OF THE EMBODIMENTS

The invention provides a flow sensor comprising an electroactive material device. A driver controls the electroactive material device to deliver heat locally to the flowing medium for which the flow is to be sensed. Temperature sensing signals are obtained and these are used to derive a flow measurement. The way the heat is dissipated relates to the flow, and it is measurable based on the temperature sensing signals.

The temperature sensing involves measuring an electrical characteristic which comprises an impedance or an impedance phase angle of the electroactive material device at at least a first frequency and at a second frequency different from the first frequency. The influences of temperature and pressure can in this way be decoupled so that the temperature can be measured at any pressure.

The invention makes use of an actuator using an electroactive material (EAM). This is a class of materials within the field of electrically responsive materials. When implemented in an actuation device, subjecting an EAM to an electrical drive signal can make them change in size and/or shape. This effect can be used for actuation and sensing purposes.

There exist inorganic and organic EAMs.

A special kind of organic EAMs are electroactive polymers (EAPs). Electroactive polymers (EAP) are an emerging class of electrically responsive materials. EAPs, like EAMs can work as sensors or actuators, but can be more easily manufactured into various shapes allowing easy integration into a large variety of systems. Other advantages of EAPs include low power, small form factor, flexibility, noiseless operation, and accuracy, the possibility of high resolution, fast response times, and cyclic actuation. An EAP device can be used in any application in which a small amount of movement of a component or feature is desired, based on electric actuation. Similarly, the technology can be used for sensing small movements. The use of EAPs enables functions which were not possible before, or offers a big advantage over common sensor/actuator solutions, due to the combination of a relatively large deformation and force in a small volume or thin form factor, compared to common actuators. EAPs also give noiseless operation, accurate electronic control, fast response, and a large range of possible actuation frequencies, such as 0-20 kHz.

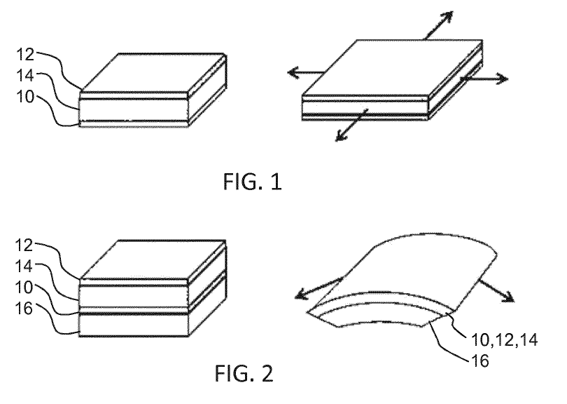

As an example of how an EAM device can be constructed and can operate, FIGS. 1 and 2 show two possible operating modes for an EAP device that comprises an electroactive polymer layer 14 sandwiched between electrodes 10, 12 on opposite sides of the electroactive polymer layer 14.

FIG. 1 shows a device which is not clamped to a carrier layer. A voltage is used to cause the electroactive polymer layer to expand in all directions as shown.

FIG. 2 shows a device which is designed so that the expansion arises only in one direction. To this end the structure of FIG. 1 is clamped or attached to a carrier layer 16. A voltage is used to cause the electroactive polymer layer to curve or bow. The nature of this movement arises from the interaction between the active layer which expands when actuated, and the passive carrier layer which does not.

The invention is of particular interest for use in a sensor which not only performs flow sensing but also performs other functions.

In particular, an electroactive polymer structure as described above may be used both for actuation and for sensing. The most prominent sensing mechanisms are based on force measurements and strain detection. Dielectric elastomers, for example, can be easily stretched by an external force. By putting a low voltage on the sensor, the strain can be measured as a function of voltage (the voltage is a function of the area).

Another way of sensing with field driven systems is measuring the capacitance-change directly or measuring changes in electrode resistance as a function of strain.

Piezoelectric and electrostrictive polymer sensors can generate an electric charge in response to applied mechanical stress (given that the amount of crystallinity is high enough to generate a detectable charge). Conjugated polymers can make use of the piezo-ionic effect (mechanical stress leads to exertion of ions). CNTs experience a change of charge on the CNT surface when exposed to stress, which can be measured. It has also been shown that the resistance of CNTs change when in contact with gaseous molecules (e.g. O₂, NO₂), making CNTs usable as gas detectors.

It has been proposed to combine the sensing and actuation capabilities of EAP devices, for example to provide pressure sensing and actuation functions, typically at separate times. An example is described in US2014/0139239.

Temporally simultaneous sensing and actuation is possible by increasing the dimensions of a device to incorporate separate dedicated sensing and actuation regions, with separate sets of electrical connections. However, this is disadvantageous in applications where small form factor is essential.

A single device may instead be used for sensing and for actuation, by providing different types of sensing and actuation signals. This approach will be described further below.

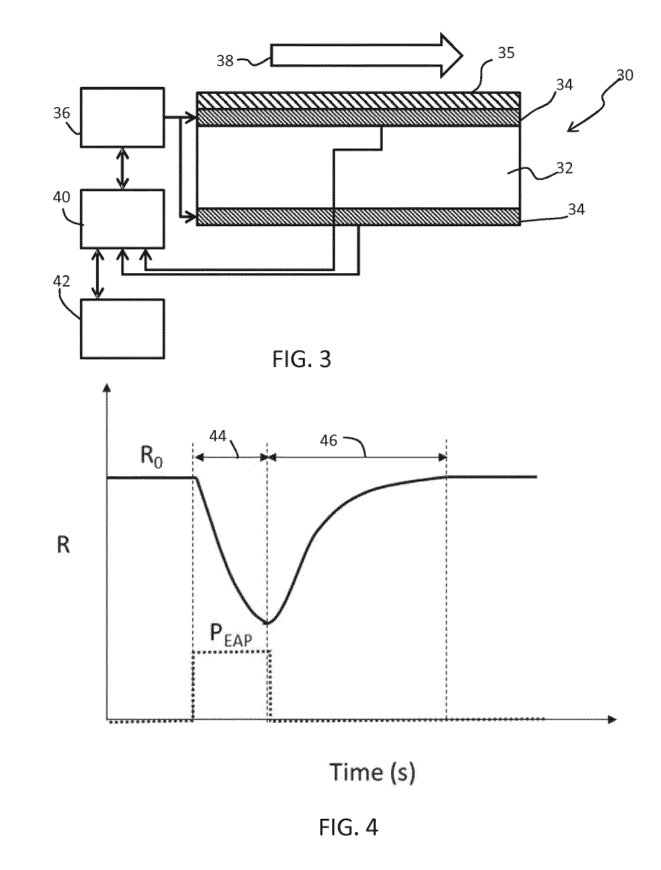

FIG. 3 shows a flow sensor comprising an electroactive material device arrangement 30. In the example shown, there is a single electroactive material layer 32 sandwiched between electrodes 34. A heat transfer layer 35 may be provided between the fluid and the remainder of the flow sensor.

A driver 36 is provided for controlling the electroactive material device arrangement 30 to deliver heat locally to the flowing medium 38 for which the flow is to be sensed.

Signals are also sensed from the electrodes 34 by a controller 40, which reads sensing signals from the electroactive material device arrangement 30. The sensing signals relate to the temperature at the electroactive material device. The way temperature can be measured by the electroactive material device arrangement 30 will be discussed further below.

A signal processing unit 42 (which may be considered to be part of the controller 40) processes the sensing signals to derive a flow measurement. Changes in the electrical state of the arrangement 30, which depend on temperature, are calibrated against the flow rate. The sensor is thus based on the principle that the flow rate influences the transport of heat away from the sensor.

Optimal heat generation by the electroactive material layer 32 is achieved by using a relatively “lossy” electroactive polymer, for example a PVDF ter-polymer, and driving it at or above its resonance frequency such that most of the electrical input energy is converted into heat. Resonance of the EAP can be optimized via its mechanical and electrical design, including geometry and fixture design.

Optimal heat flow from the sensor to the medium can be implemented in two different ways. Firstly, if the cooling power of the medium is very high, it might be beneficial to decrease the heat transfer coefficient from the electroactive material layer to the medium, for instance by applying a thermal isolation layer, such that the EAP can hold enough heat to achieve a measurable temperature, and to delay the cooling rate in order to differentiate between different cooling rates. Secondly, if the cooling power of the medium is very low, it might be beneficial to take opposite measures in order to optimize the measurement sensitivity and accuracy. Thus, the design of the heat transfer layer 35 takes into account the nature of the medium and expected flow rates.

The heat transfer layer 35 may also function as a seal to enable operation in fluids.

There are various ways to control the heating and measure the temperature in order to derive the flow rate.

A first approach is based on determining the cooling rate after a finite electrical power input. This operates as an open loop system, for example suitable for static or slowly varying flow rates.

FIG. 4 shows a plot of impedance R versus time for this control approach.

Before starting the measurement, the electroactive material layer may be brought into an electrical reference state, for example by applying one or more reset pulses.

A reference measurement is carried out to quantify the electrical state R₀ of the electroactive material layer corresponding to a baseline temperature.

During a short time interval (e.g. 10 seconds) the actuator is driven at or above its resonance frequency to generate heat. This creates a heating cycle 44, during which electrical power P_{EAP} is delivered. This time interval is predetermined in order not to overheat the system or its environment. For instance a 45° maximum temperature is appropriate for a sensing operating in a blood flow. As an alternative the electroactive material layer can be heated up to a predetermined temperature using feedback control.

Immediately after this time interval 44, the temperature decay during a cooling cycle 46 is monitored via an electrical parameter which is a function of the temperature.

The time needed to reach the reference state R₀ again, corresponding to the original temperature, correlates to the heat transfer rate, which correlates to the flow rate of the medium. This time is the duration of the cooling cycle 46.

A calibrated formula or a look-up table may then be used to convert the cooling time period to a flow rate.

A second approach is based on determining the steady state temperature during a constant electrical power input. Again, this is an open loop system suitable for static or slowly varying flow rates.

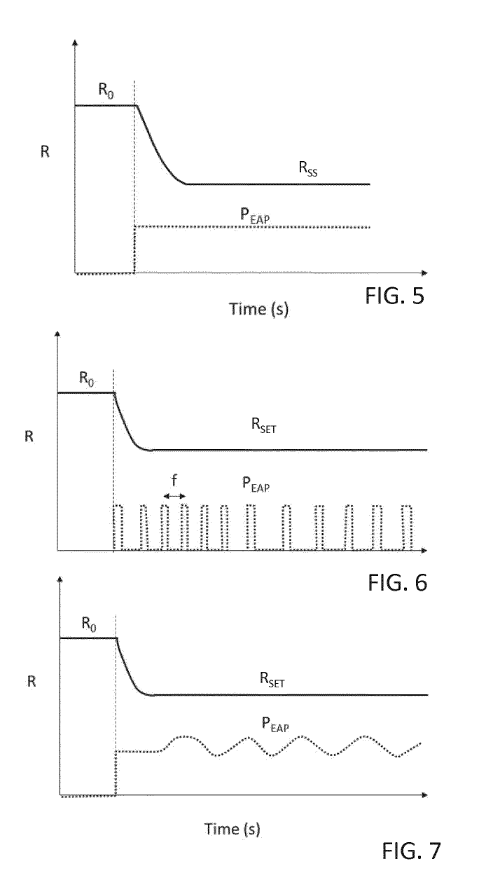

FIG. 5 shows a plot of impedance R versus time for this control approach.

Before starting the measurement, again the electroactive material layer may be brought into an electrical reference state, for example by applying one or more reset pulses.

A reference measurement is carried out to quantify the electrical state R₀ of the electroactive material layer corresponding to a baseline temperature.

A constant electrical power input P_{EAP} is then applied. The steady state temperature, with a corresponding steady state electrical parameter R_{SS}, depends on the flow rate. A calibrated formula or a look-up table may then be used to convert the steady state electrical parameter to a flow rate.

A third approach is based on determining the required power input to maintain a constant temperature. This is a closed loop control system which is particularly suitable for varying flow rates.

FIG. 6 shows a plot of impedance R versus time for this control approach.

As in the examples above, before starting the measurement, the electroactive material layer may be brought into an electrical reference state, for example by applying one or more reset pulses.

A reference measurement is carried out to quantify the electrical state R₀ of the electroactive material layer corresponding to a baseline temperature.

The electrical heating power P_{EAP} is not constant, but is varied using a closed loop feedback approach to maintain a constant value R_{SET} of the electrical parameter. This is suitable in a varying flow rate if the response time of the closed loop system is fast enough.

In the example of FIG. 6, the electrical power is provided as a series of constant voltage pulses, of which the frequency f is varied to keep the parameter R constant.

FIG. 7 shows an alternative approach in which the power P_{EAP} is continuously adapted to keep the parameter R constant.

The examples above have one sensor. An alternative is to provide a calorimetric flow sensor (for slowly varying flows), in which three sensors are used. One device heats at a constant electrical power, and there is a sensor on each side to measure the temperature. The middle heating device may instead apply a sinusoidal or block wave heating profile. The phase delay between the temperature of the heater and the sensor elements is derived in order to determine by the local flow rate.

The examples above show how flow rate sensing is possible using an electroactive material device. The device may still perform other functions, such as the typical pressure sensing or actuation functions of an electroactive material actuator or sensor.

A full combination of functions is to provide flow rate measurement, pressure sensing and actuation.

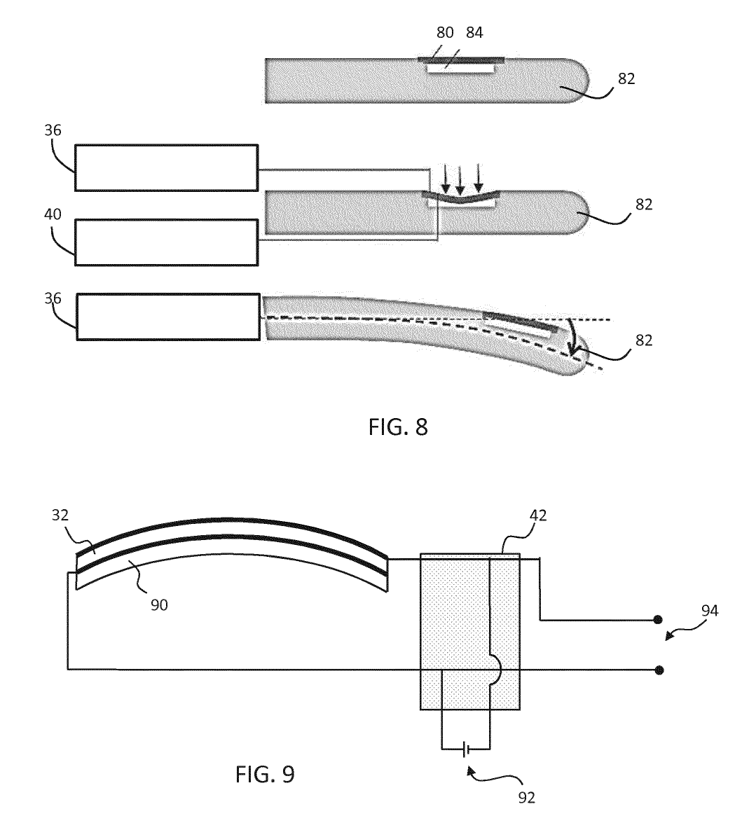

FIG. 8 shows the electroactive material device 80 formed in the tip of a catheter 82, suspended over a cavity 84. The device may in the same way be provided along or at the tip of a guidewire, such as a catheter guidewire or a stent delivery guidewire. To measure flow and pressure as shown in the middle image, the device is driven by driver 36 to deliver heat and then to operate at multiple frequencies as explained below, with resistance or impedance measured by controller 40. For actuation as shown in the bottom image, a DC (or low frequency) signal is applied by driver 36.

For flow pressure sensing, the sag induced in the device 80 depends on the pressure. Actuation of the device may be performed to induce bending, for example for steering, scanning, or motion compensation. The pressure sensing may then comprise blood pressure sensing.

The actuator may be driven with an AC signal superimposed on a DC signal for simultaneous sensing (of temperature and optionally also pressure) and for actuation. The device may be used for intravascular devices and applications.

It is well known that flow varies across a tube such as a blood vessel—flow being lowest at the wall of the vessel and highest in the center. For this reason, to obtain a representative measurement of the blood flow it is highly beneficial to know the position of the flow sensor in the vessel. Several approaches can be taken to improve the measurement, which involve laterally changing the position of the sensor across the vessel.

The use of actuation such as in the arrangement of FIG. 8 enables lateral movement to be controlled by applying a DC voltage signal to the electroactive material device. The flow measurement may then be repeated at several positions across the vessel and the highest recorded cooling rate be interpreted as the flow rate of blood in the vessel. The sensor may be continuously scanned across the vessel (for example at a frequency of around 1 Hz) during the measurement. In this manner, a flow rate averaged across the vessel is obtained, which is representative for the vessel. In particular, where only changes in flow rate along a vessel are required (instead of absolute rates) it may be particularly advantageous to apply the continuous scanning approach.

The way in which the sensing signals may provide a temperature measurement will now be described.

The sensed parameter is an impedance of the electroactive material sensor, and in particular the impedance may be measured at at least first and second different frequencies. From these measurements a temperature at the sensor as well as (if desired) an external pressure or force applied to the sensor can be determined. The sensor can thus be used as a pressure sensor and as a temperature sensor.

In FIG. 9 is shown a schematic illustration of a simple first arrangement for an actuator and temperature sensor device.

The electroactive material actuator again comprises an electroactive material layer 32 disposed on a lower carrier layer 90 and is electrically connected via the signal processing element 42 with a first (DC) drive signal input 92 and a second (AC) drive signal input 94. The first drive signal input 92 is for application of a (relative) high power actuation drive signal. The second signal input 34 is for application of a (relative) low power alternating sensing signal, and in particular at two different frequencies, as will be discussed below.

The signal processing element 42 superposes the first and second drive signals to form a third combined drive signal, which is then applied across the device.

The signal processing element may in examples comprise a number of component elements for performing, for example, signal analysis functions, signal coupling and decoupling functions and/or signal generation functions. In the latter case, the first and second drive signal inputs 92 and 94 may be encompassed within the processing unit 42 itself, the processing unit comprising elements for generating AC and/or DC signals and, in some cases, elements for analysis of electrical parameters of one or both signals.

The electrical connections of the arrangement of FIG. 9 are shown connected to electrodes at the top and bottom planar surfaces of the electroactive material layer. Flexible electrode arrangements may be used for this purpose. Application of DC and/or AC voltages to the electrodes allows the generation of an electric field across the electroactive material layer which stimulates a corresponding deformation.

Although the first drive signal input 92 in the arrangement of FIG. 9 comprises a DC input, in alternative arrangements, this input may comprise an AC drive signal input. In either case, the relative power of the actuation drive signal significantly exceeds that of the applied sensing signal. In the case that both signals comprise AC signals, the maximal amplitude of the sensing signal (applied at 94) may be less than 10% of the maximal amplitude of the actuation drive signal (applied at 92), for example less than 1% of the maximal amplitude of the actuation drive signal. In the case that the sensing signal comprises an AC signal, and the actuation signal comprises a fixed amplitude DC bias signal, the maximal amplitude of the AC signal may be less than 10% of the fixed amplitude of the DC bias signal, for example less than 1% of the fixed amplitude of the DC bias signal.

For the example of FIG. 9, the third combined signal generated by the signal processing element 42 comprises a high frequency, low-amplitude AC signal superposed atop a high amplitude DC bias signal.

As described in preceding sections, the application of a DC bias of sufficient amplitude across a layer of electroactive polymer stimulates an expansion of the polymer layer. If the layer is coupled with a passive carrier layer 90 the expansion of the polymer results in a deformation, for example a bending or warping, of the overall structure, which may be used to provide an actuation force. In FIG. 9, the actuator structure is shown in an ‘active’ or ‘actuated’ state, wherein a DC bias is being applied of sufficient magnitude to cause a deformation of the structure. As is well known, the extent of expansion varies in relation to the magnitude of the electric field/electric current applied across the device. Hence by varying the amplitude of the DC bias, deformation of differing degrees/extent may be induced, and differing magnitudes of actuation forces applied (or differing amounts of actuation work done, for example).

The high frequency AC signal superposed atop the DC bias also stimulates a mechanical deformation response in the material, but a deformation response which is periodic, rather than fixed (i.e. an oscillation). However, since the maximal amplitude of the high frequency signal is significantly lower than the amplitude of the DC bias signal (for example two orders of magnitude lower than that of the DC bias signal, for example, 1% of that of the DC signal), the corresponding displacement amplitude of the stimulated deformation is effectively negligible compared to the primary actuation displacement. Hence the accuracy and stability of the actuation is not affected by the superposition of the sensing signal.

The overlay of a low-amplitude oscillation signal on top of the DC bias allows for an electrical feedback mechanism to be incorporated within the primary actuator driving mechanism itself. At certain frequencies, in particular at frequencies which match or are harmonic with the mechanical resonant frequency of the actuator structure, a small mechanical standing wave is established in the material of the actuator. This in turn influences the electrical characteristics of the material. When the sensing signal is driven at the resonance frequency of the material, the corresponding impedance of the material is lower (compared to when driven at non-resonance) due to the mechanical vibration being in-phase with the electrical driving signal.

The mechanical resonance frequency of a structure is the frequency at which a structure will naturally tend to oscillate, upon being displaced from its equilibrium position, and is determined by intrinsic structural properties of the structure (e.g. geometry, size, shape, thickness etc.). The mechanical oscillation of the EAP structure will not necessarily follow the drive frequency of the electrical signal applied to it, but will tend to fall back to its natural resonance frequency, with the drive frequency interfering with that oscillation either constructively or destructively, depending upon the degree to which the driving frequency is either out of phase or in phase with the natural oscillating frequency (resonance frequency).

When the high-frequency signal is driven at the anti-resonance frequency of the electroactive material structure (i.e. the first harmonic of the resonance frequency), the impedance of the electroactive material is higher, due to the mechanical vibration of the material being out of phase with the oscillation of the drive signal (the electrically induced mechanical strains are out of phase with the electrical excitation). In other words, whenever, for instance, a positive current is being applied to the electroactive material by the drive signal, the out of phase mechanical strains are at the same moment inducing a current in the opposite direction (i.e. out of phase behavior). In the ideal (model) case these opposing currents cancel each other out, and no current can flow at all (i.e. infinite impedance), but in real-world scenarios no full cancellation occurs and this effect is measured as an (effective) higher resistance of the electrical current (i.e. higher impedance). In particular, when the signal is driven at the anti-resonance frequency of the actuator material, the impedance of the electroactive material is at a maximum.

The relationship may be further understood by considering equation (1) below. The impedance of an ideal electroactive material at resonance and anti-resonance depends on the particular type or mode of deformation. It is most common to bring the electroactive material into lateral resonance (i.e. length or width). The impedance is governed by the dielectric properties of the material and the electromechanical coupling and electrical and mechanical losses. For simplicity, when ignoring the electrical and mechanical losses, for an electroactive material layer with length l, width w and thickness t, deforming in lateral extension, the impedance is given by:

$$Z ( ω ) = \frac{1}{i ω \frac{lw}{t} {ɛ}_{33}^{T} [ {( {k}_{31} )}^{2} \frac{tan ( \frac{ω l}{2} {( ρ {s}_{11}^{E} )}^{1 / 2} )}{\frac{ω l}{2} {( ρ {s}_{11}^{E} )}^{1 / 2} γ {α}^{( E )}} + 1 - {( {k}_{31} )}^{2} ]}$$

where ε^{T}₃₃ is the dielectric constant, k₃₁ is the lateral electromechanical coupling factor, p is the density of the EAP and s^{E}₁₁ is the compliance in the lateral direction. At anti-resonance frequency, ωₐ, tan

$$( \frac{ω l}{2} {( ρ {s}_{11}^{E} )}^{1 / 2} ) = 0$$

and Z is highest.

A real electroactive material has losses and can be modeled or represented by a capacitor with a resistor in series, the resistance of which is greatest at the anti-resonance frequency. In the descriptions which follow, therefore, ‘impedance’ and ‘series resistance’ (Rs) may be used interchangeably with reference to the device. However, series resistance is to be understood in this context as referring simply to a model in which the actuator/sensor is represented electronically by a capacitor in series with a resistor, having resistance Rs.

In consequence of the above-described relationship between impedance and resonance, when the drive signal is being driven at the anti-resonance frequency, any small deviations which occur in its frequency away from anti-resonance will be detectable in a corresponding sharp drop-off the in measurable impedance of the EAP structure. It is this physical effect which allows mechanical sensing to be achieved.

Application of load (i.e. pressure or force) to the structure results in a dampening of any resonance effects which are occurring within the material. If the drive signal is oscillating at the anti-resonance or resonance frequency of the material when the load is applied, the dampening effect will be identifiable within real-time measurements of the EAP impedance (i.e. series resistance Rs), as the sudden cessation of resonance will effect a consequent sharp decline in the impedance. Hence by monitoring the impedance of the structure over time, while the actuator is in operation (for example by monitoring the voltage and current of the high-frequency signal over time), pressures and loads applied to the structure can be sensed, and in some cases quantitatively measured (as will be described below).

The link between impedance on the one hand, and the phase difference between the electrical drive frequency of the signal and the mechanical oscillating frequency of the material on the other, allows for highly sensitive measurement of applied mechanical forces to the EAP to be achieved through the monitoring of electrical properties of the drive signal only. This hence provides a highly simple, straightforward and efficient means for achieving simultaneous actuation and sensing using a single EAP device. Moreover, embodiments allow simultaneous sensing and actuation over the same region of EAP structure (i.e. spatially simultaneous sensing and actuation). This means that a device performing both functions can be made with a much smaller form factor, without sacrificing sensitivity or resolution of sensing for example. Moreover, only a single set of connections is require to be provided to the device (as opposed to two or more sets of connections, one for each dedicated sensing or actuation region) which is advantageous in terms of cost and reduced complexity, and in cases where watertight connections are required for example (for instance in shaving/catheters/oral healthcare) and/or where an array of actuators/sensors is to be constructed.

By suitable selection of sensing signals and with suitable signal processing, the sensing provides temperature as well as load sensing, which is then used to derive flow rate information in the manner explained above.

In particular, measurement signals of least first and second different frequencies are generated, and the signal processing element 42 is used to measure one or more electrical characteristics of the actuator 30 at the two measurement frequencies. In this way, a temperature at the actuator and an external pressure or force applied to the actuator, may both be determined.

If only temperature information is needed (which is then used to derive flow rate information), then of course the force calculations are not needed. However, the use of two measurement frequencies enables the temperature effects to be decoupled from the external force effects.

The frequency of the high-frequency sensing signals may each typically be in the range of 1 kHz to 1 MHz, depending on the particular geometry of the actuator. Note that in the case that the actuator drive signal comprises an AC drive signal, the frequency of this signal is significantly lower than that of the alternating sensing signal. The (low frequency) actuation voltage in this case may for example be at least two orders of magnitude lower than the high frequency signal voltage, to avoid interference of the actuator signal with the measurement signal.

As explained above, at the anti-resonance frequency, the measured impedance is higher due to the out-of-phase mechanical vibration. In particular, the series resistance (Rs) of the actuator is at a local maximum at this frequency. In one implementation, this frequency is used as a first one of the measurement frequencies. Another measurement frequency is defined which is outside the electromechanical coupling frequency range, and this is used as the second measurement frequency.

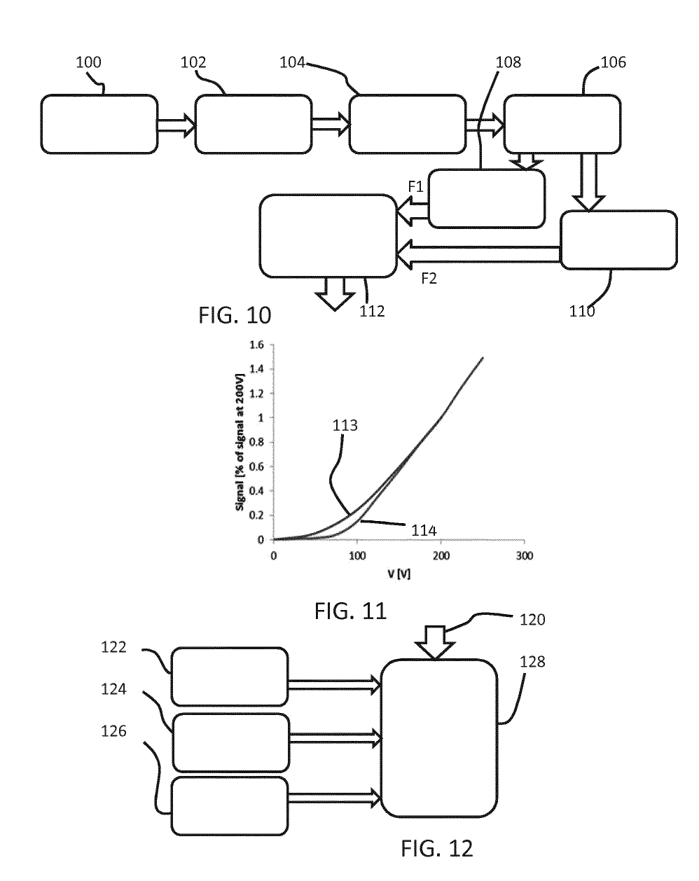

A calibration process may be used to determine the frequencies to be used and for determining a relationship between measured resistance and applied load at said determined resonant frequency. FIG. 10 shows one example.

A first frequency sweep 100 is performed, at an applied DC bias of 0V, and resistance responses measured. The equivalent series resistance of the actuator is thereby measured at the different frequencies to obtain an impedance versus frequency function, with no actuation signal present.

A fixed DC bias is then applied in step 102, preferably corresponding to a desired actuation state of the device. At this time, there may be no load applied to the device.

A second frequency sweep is then performed in step 104 at the fixed non-zero DC bias, and corresponding resistance values recorded. The equivalent series resistance of the actuator is again measured at the different frequencies to obtain an impedance versus frequency function, with an actuation signal present.

The results of the two sweeps are then compared in step 106 to determine the difference in the obtained resistance values for each across the range of frequencies.

In step 108, the first frequency for which the measured resistance values differ by the greatest amount is determined and the anti-resonance frequency thereby directly identified.

In step 110, the second measurement frequency is defined. It is a frequency at which the difference is negligible. Thus, it is a frequency at which the electrical characteristic is constant with respect to load.

Note that steps 100 to 110 may be in some cases repeated for as many DC voltages as are desired, for example to gather data relating to a plurality of different actuation positions, in the case that variable actuation extent is to be employed in the operation of the device.

For a sensor-only device, there will be a single actuation, which brings the sensor into an actuated state at which it is ready to perform sensing. Thus, only one driven calibration is needed.

The sensor could for example be set into a position and used from then on as a sensor only. This may be considered to correspond to a single actuation level used for making multiple sensing measurements. A sensing function may be used with a DC bias within a certain range. However, this range may include DC bias voltages for which there is no physical actuation, but there is nevertheless sensitivity to an applied load. In particular, the actuation curve (actuation versus applied voltage) is non-linear with a threshold voltage below which physical actuation does not start. In this case, the sensing function is enabled even without physical deformation, although the sensed signal will be smaller than for a larger DC bias.

FIG. 11 shows a plot of the signal strength for sensing a fixed load at different actuation voltages, as plot 113. Plot 114 shows the actuation level for those actuation voltages (with arbitrary scale). It can be seen that the sensitivity increases more rapidly than the actuation for voltages increasing from an initial zero level.

A typical DC bias range for sensing only may for example be in the range 40V to 50V, or 40 to 75V, where sensitivity is above zero but actuation is still zero or close to zero (respectively).

In step 112 of FIG. 10, calibration data for the impedance value is derived, in the form of series resistance across the device versus applied load, for a fixed DC bias voltage, and a fixed AC signal frequency—equal to the anti-resonance first frequency.

Furthermore, an impedance value is obtained for each temperature in a range of interest and for each possible actuation signal. At the second frequency, an impedance value is obtained for each temperature in a range of interest, for each possible actuation signal, and for each possible load.

Thus, in step 112, there are multiple measurements at different temperatures and with different load applied. This calibration process takes place in the factory and a lookup table is generated for Rs at frequency 1 and frequency 2 for variable applied load and temperature. At each temperature, the full range of loads is measured. This lookup table is used as reference during use.

In this way, the actuator is calibrated for the impedance versus load for each applied voltage (if there are multiple applied voltages) and at each temperature point within the temperature range.

During actuation, the measured impedance value at the first frequency in combination with the applied voltage gives a measure for the force on the actuator and the impedance value at the second frequency gives a measure of the temperature of the electroactive material actuator. The displacement amplitude of the high frequency (sensor) signal is negligible compared to the actuation displacement, so it will not interfere with the actuation in terms of accuracy or stability.

As is clear from the discussion above, the actuation is optional.

FIG. 12 shows the method which is used during use of the actuator. The calibration data is received as represented by arrow 120. Step 122 involves measuring the impedance at the first calibration frequency. This is used for load (i.e. pressure or force) sensing. Step 124 involves measuring the impedance at the second calibration frequency. This is used for the temperature sensing.

During these measurements, the higher amplitude actuation signal is applied in step 126. It will be a constant for a sensor only implementation or it will be variable for a sensor and actuator. Step 128 involves deriving the load on the actuator and the temperature.

These two parameters may be provided as separate outputs from the system. Alternatively, the temperature information may be used internally by the system to provide temperature compensation of the sensed load.

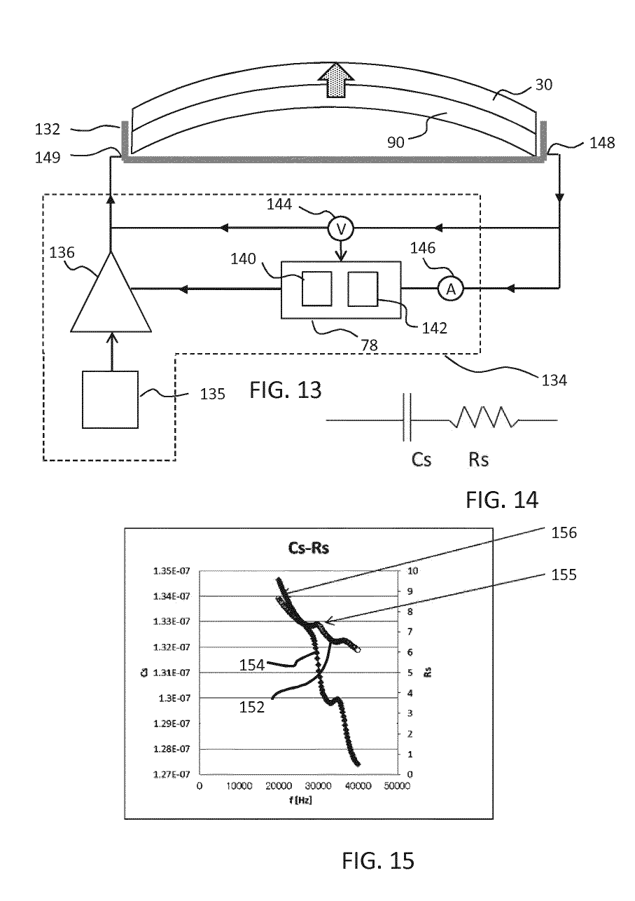

A first example will be described in more detail, based on a DC actuation signal, as shown in FIG. 13.

As explained above, the EAP actuator has an electroactive material (e.g. EAP) layer 32 and passive carrier layer 90 and is held within a housing 132, and is electrically coupled with a signal drive mechanism 134. The drive mechanism in the example of FIG. 13 comprises both signal generation elements (drive elements) and signal processing and analysis elements (sensor elements).

An actuator control element 135 generates a high-amplitude actuator drive signal (for example a fixed DC bias voltage) which is transmitted to a signal amplifier device 136. A sensor control element 138 comprises both a driver element 140 for generating the sensor signals, and a processing element 142 for analyzing electrical properties of the sensor signals after passage across the actuator. To this end, the drive mechanism 134 further comprises a voltmeter 144, connected across the EAP actuator, and an ammeter 146 connected in series between the outgoing electrical terminal 148 of the actuator and the sensor control element 138. The voltmeter 134 and ammeter 136 are both signally connected with the sensor control element 138, such that data generated by them may be utilized by the processor 142 in order to determine an impedance of the actuator (that is, the equivalent series resistance Rs where the device is modeled as an ideal capacitor with a resistor in series, i.e. the real part of the complex impedance).

Drive signals generated by the actuator control element 135 and sensor control element 138 are superposed by the amplifier element 136, either in advance of their combined amplification, or after their independent amplification. In some examples, the amplifier element 136 might be replaced simply by a combiner. In this case actuator control element 135 and sensor control element 138 may be adapted to amplify their generated actuation and sensing signals locally, in advance of outputting them to the combiner.

The combined drive signal is then transmitted to the ingoing terminal 149 of the EAP actuator. The high amplitude DC component of the combined drive signal stimulates a deformation response in the actuator.

For the most reproducible (i.e. reliable/accurate) results, the EAP may be clamped in position. For example, the actuator may be clamped within housing 132, and the housing then positioned so as to align the device with the target actuation area.

The low-amplitude AC component of the drive signal stimulates a low amplitude periodic response in the EAP layer 32, for example oscillating the structure at its resonant or anti-resonant frequency.

The voltage of the combined drive signal and the resulting current are fed to sensor control element 138. Typically the AC currents may be in the range of 0.1 mA to 1 mA, but may be up to 10 mA. Higher currents may cause too much heating.

In some cases, the drive mechanism 134 may further comprise one or more signal decoupling elements, for example a high pass filter, for the purpose of isolating high-frequency components for analysis by the processing element 142 of sensor control element 138.

The processing element 142 of sensor control element 138 may use measurements provided by voltmeter 144 and ammeter 146 in order to determine a series resistance across the actuator, as experienced by the applied drive signal(s). The series resistance may be determined in real time, and monitored for example for sudden changes in resistance, which as explained above, may be used to indicate the presence and magnitude of loads and pressures applied to the actuator.

The EAP actuator has an approximate equivalent circuit of a series capacitor Cs and resistor Rs as shown in FIG. 14.

The sweep explained above, which is used to determine the anti-resonance frequency (the point of highest sensitivity), is shown in FIG. 15.

The measured series resistance (in Ohms) is shown on one y-axis, the measured capacitance (in Farads) is shown on another y-axis and the sensor signal frequency (in Hz) on the x-axis.

Plot 152 is the resistance and plot 154 is the capacitance. For this sample, a frequency of around 29.8 kHz is determined as the anti-resonance frequency as a result of the local resistance peak shown as 155. A frequency away from the point is selected as the second frequency, such as point 156 at 20 kHz. The plots are for a bias voltage of 200V.

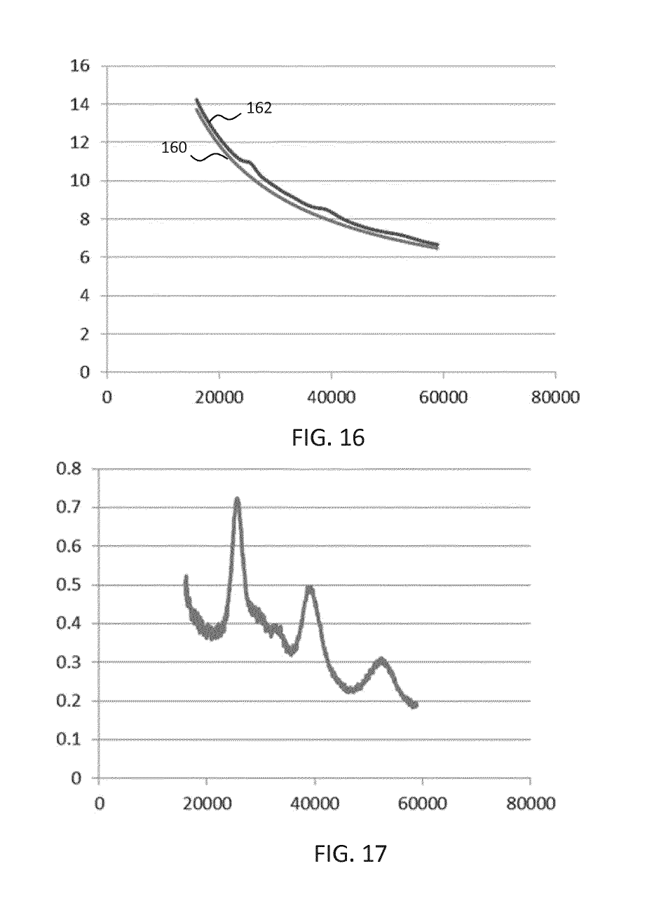

As explained above, the peaks are most easily determined by comparing plots. FIG. 16 shows a resistance measurement for a 0V sweep as plot 160 (which shows no variation about the primary curve which reflects simply a capacitive complex impedance function) as the AC frequency is varied. At 0V bias, there is little or no coupling, and hence zero (or unmeasurably small) deformation response in the material to the AC signal. The 0V bias sweep hence provides a convenient baseline against which to compare an AC frequency sweep at a higher (actuation inducing) DC voltage. Plot 160 is the sweep with an applied DC bias.

The anti-resonant frequency of the device may be identified by finding the AC frequency for which the difference between the measured resistance values for the two DC voltages is the greatest.

In FIG. 17 is illustrated more clearly the difference between the two signal traces, with difference in measured resistance on the y-axis and corresponding sensor signal frequency on the x-axis. The two larger jumps in resistance are clearly visible in this graph, with the larger of the two being the jump occurring at anti-resonance.

Although a DC bias of 0V is used for the first sweep in this example, in alternative examples a different (non-zero) first bias might be used. In this case, depending on the magnitude of the first voltage, the first sweep may indicate variations or peaks about the central curve. However, the anti-resonance frequency may still be found by identifying the frequency for which the difference between the measured resistance values for the two DC voltages is the greatest.

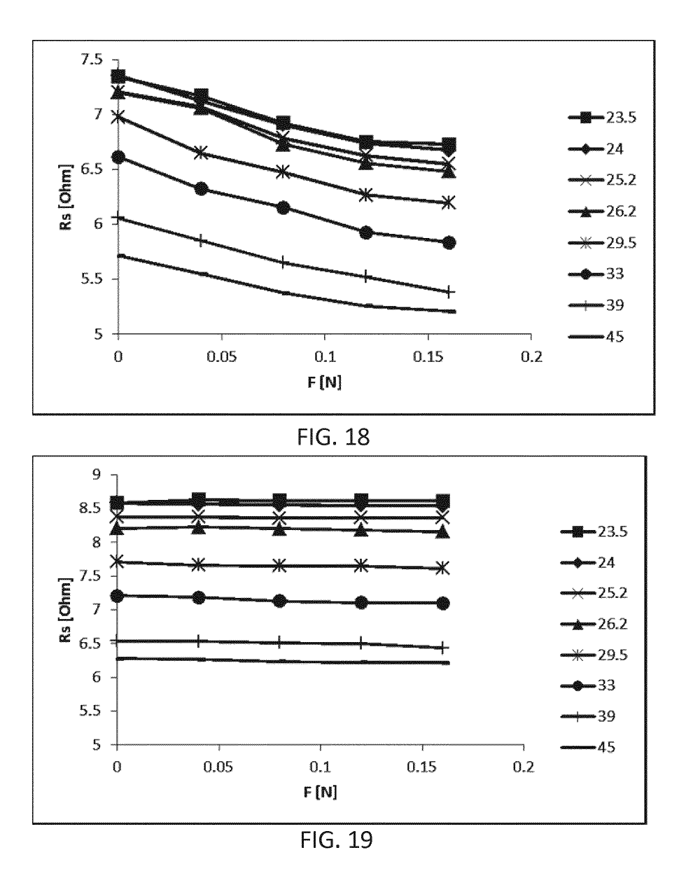

The load also has an influence on the series resistance of the actuator, by damping the resonance-anti resonance behavior. This is shown in FIG. 18 which plots the resistance Rs at anti-resonance measured on an actuator with 200V bias against the load. Each plot is for a different temperature, and the temperature offset drift is visible.

At the second frequency (outside resonance coupling range) there is no influence of the electro mechanical coupling. At this frequency the resistance is only a function of temperature as shown in FIG. 19, which plots the resistance against the load. The resistance is plotted for the off resonance frequency (20 KHz) again measured for an actuator with 200V bias.

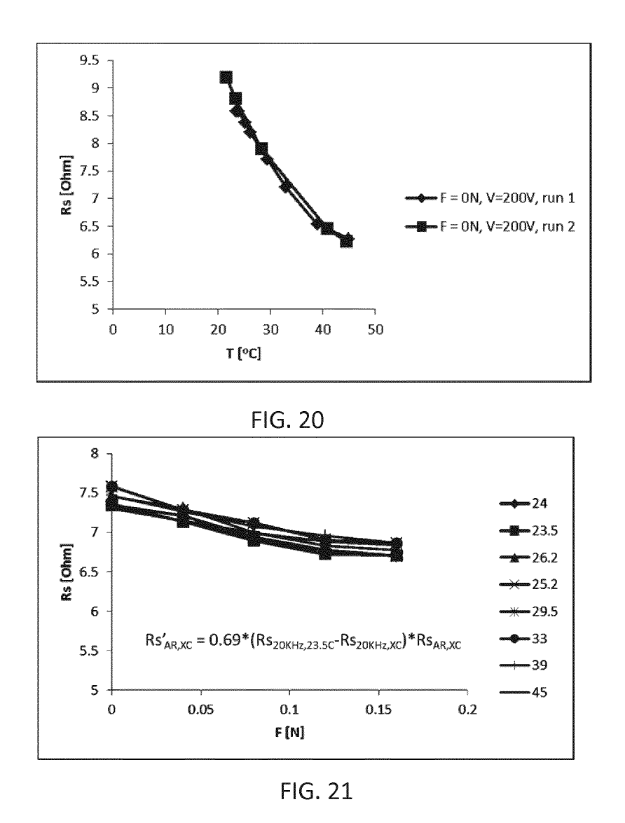

The temperature offset drift is visible, but there is no influence from the applied load. As shown in FIG. 20, the temperature signal is reproducible because FIG. 20 plots the resistance versus the temperature for zero load, for two runs.

As explained above, the temperature dependency of the signal is used to derive flow rate information.

The temperature signal can also be used for compensation of the actuator signal, to improve the accuracy of the load sensor. In FIG. 21, the compensated resistance value as a function of load is given for 8 different temperatures from 23 to 45 degrees. The average difference between 23 degrees and 45 degrees is now 3.8% instead of 29% for non-compensated measurement.

The example above is based on a DC actuation signal. In a second example, there is a low frequency AC actuator signal. For low frequency AC actuation, the actuator is loaded electrically by a low frequency AC voltage and a small signal, high frequency AC voltage. The small amplitude, high frequency voltage is used for measurements and is superimposed on the low frequency AC actuator signal. The low frequency AC actuator voltage causes a deformation in the EAP which can be used for actuation purposes.

The low frequency actuation voltage preferably has a frequency at least 2 orders of magnitude (i.e. <1%) lower than the high frequency signal, to avoid interference of the actuator signal with the measurement signal.

In a third example, a frequency scan is not required to calibrate the system. This enables the system complexity and cost to be reduced. However, robustness and sensitivity can still be ensured. In production, the (anti-)resonance frequency (fᵣ) of an actuator will be tightly controlled so a predetermined set of 2 frequencies per temperature point within the temperature range is known a priori, thus a measurement at these two predetermined frequencies will always be indicative of load on the actuator (frequency 1) and temperature (frequency 2).

In a fourth example, a sensing device or an actuation and sensing device may be provided comprising a plurality of devices according to the above described examples, for example arranged in an array, or other desirable layout/shape. In examples, the plurality of devices may be provided such that each has a unique mechanical resonance frequency fr. In this way, on application of high frequency sensing signals to the array of devices, the characteristic (unique) resonance frequency of each device may be used to determine which actuator in the array is being stimulated as a sensor, i.e. to give the position of the sensor/actuator in the array.

For example, a common drive signal may be applied across all devices in the array, the common signal comprising a sequential series of signals of different frequencies (i.e. the known different resonance—or anti-resonance—frequencies of the devices). If the time-sweep of frequencies is faster than the sensor input, then a corresponding drop (or rise) in impedance will be detectable across the devices only for that frequency corresponding to the specific device which is stimulated, i.e. measured impedance will drop as the frequency sweep moves into fr corresponding to the stimulated device, and then rise again (or vice-versa) as the sweep moves out of fr. In such a system, fᵣ (or Rs) can be used to identify which actuator is being used as a sensor i.e. to give the position of sensor/actuator in the array. The example above makes use of impedance measurement to determine the applied load. Instead of detecting the (change of) the series resistance, the change in anti-resonance frequency may be detected to derive the corresponding feedback signal.

Alternatively, instead of detecting the (change of) the series resistance (or change in anti-resonance frequency) the change in phase may be determined, in particular the phase angle of the complex impedance. The change in series resistance Rs is relatively small. To improve sensitivity, it may be combined with another dependent variable.

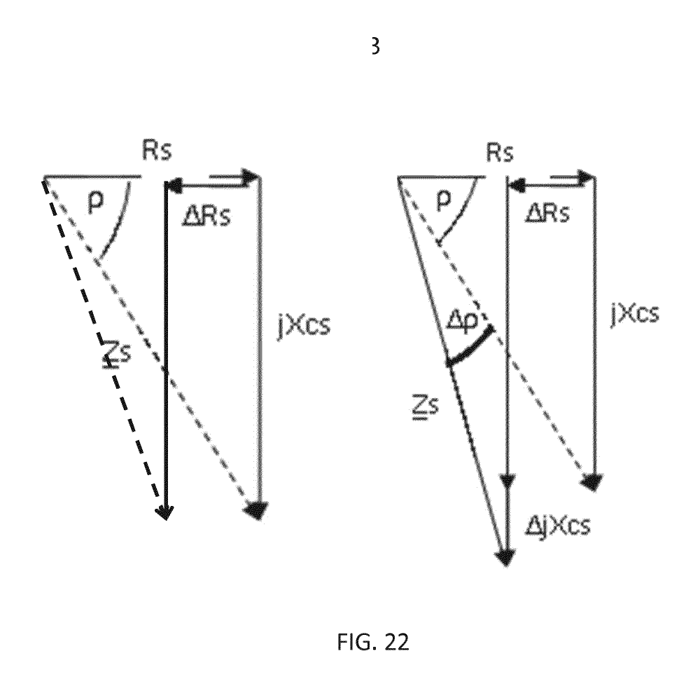

In FIG. 22, a change in Rs is shown on the left, and a change in Cs and Rs is shown on the right.

The right image shows how the phase angle of the complex impedance changes by an increased amount (Δρ) in response to a decrease in the real impedance part and an increase in the imaginary impedance part. The phase can be detected by measuring the change in phase between current and voltage. Especially, if EAPs have thin layers, the effect of changes in the imaginary part of the impedance (jXcs) may become dominant. Indeed, any measurements correlated to the complex impedance can be used to signify the loading of the actuator.

The sensitivity of the temperature sensing function may be tuned by suitable selection of the composition of the polymers (of the EAP actuator/sensor) used. The composition may be tuned to obtain the highest sensitivity of the sensor to the desired working temperature.

For example, in a (PVDF-TrFE-CTFE) polymer material, this can be achieved by varying the CTFE content.

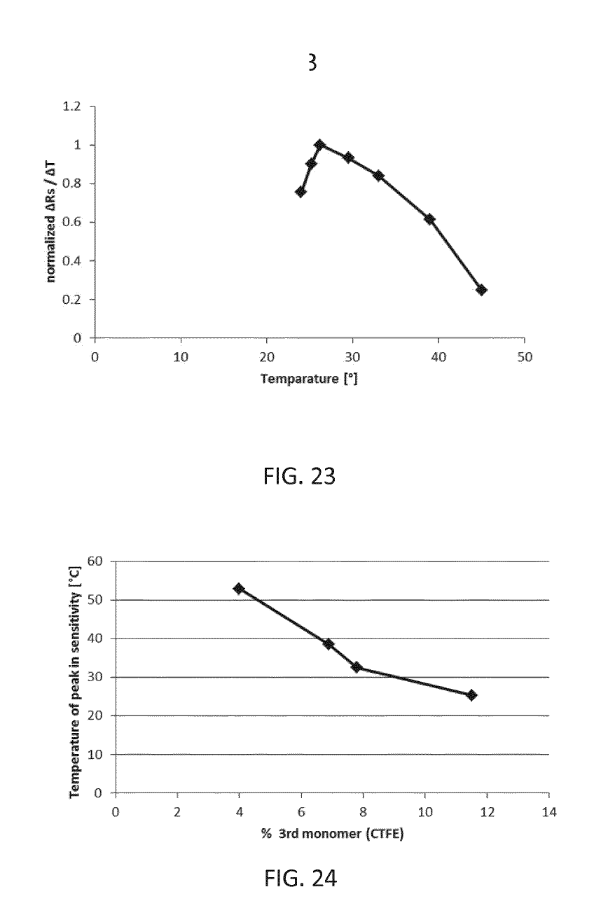

FIG. 23 shows the sensitivity of an example material (PVDF-TrFE-CTFE) with a certain composition versus temperature, and it shows a maximum sensitivity at 26 degrees Celsius. The example material has 10% CTFE content.

FIG. 24 shows the relationship between the suitable working temperature and CTFE content of the (PVDF-TrFE-CTFE) polymer, and shows the temperature at which the temperature sensitivity is highest versus the percentage of the CTFE content. As shown, a higher CTFE content gives rise to a reduced temperature at which the sensitivity is highest. For example a polymer with 7% CTFE may be used for in-body applications where the temperature is higher than for an indoor sensor at room temperature.

The electroactive material (e.g. EAP) is used as a heater in the device described above. It will now be shown that that sufficient heating can be obtained. Two conditions are considered; a static air condition (with low cooling capacity) and a circulating blood condition (with strong cooling capacity). A desired temperature increase is for example 5° C.

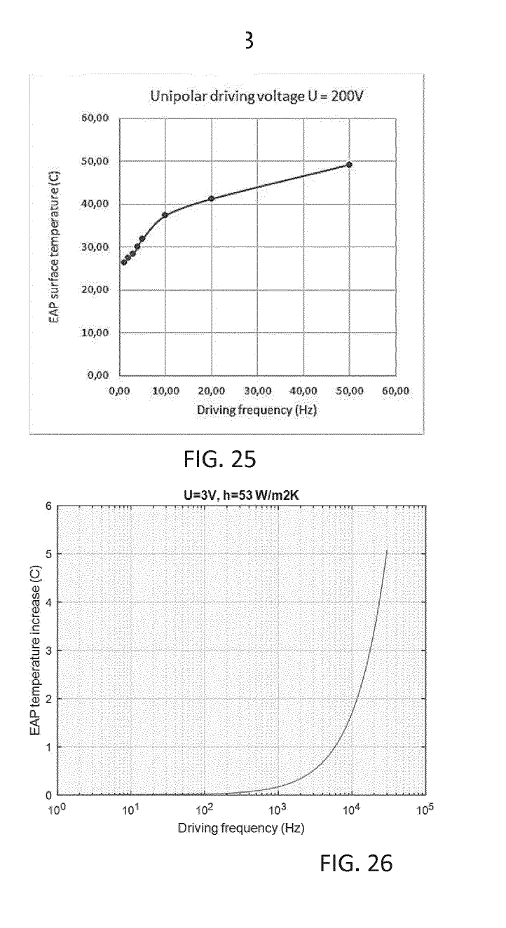

It is known that EAP actuator heats up easily in static air. When driven at relatively low frequencies (1-50 Hz) and high voltages (150-200V) the temperature increase of an actuator can be more than 10° C. within a few seconds, as shown in FIG. 25. FIG. 25 plots the maximum EAP surface temperature (y-axis) in static air as a function of the driving frequency (x-axis), measured with an infrared camera. The maximum temperatures are reached within 10 seconds.

Basic equations for heat generation and convective heat transfer can be used to estimate the heat transfer coefficient in air using the measurements above. The convective heat transfer from a body to a medium is described with:

Q=h·A·(Tₑₐₚ−T_{flow}) (1)

where Q is the heat flow (J/s), h is the heat transfer coefficient of the system (J/m² sK), A is the area (m²), and Tₑₐₚ and T_{flow} are the temperatures (Celsius or Kelvin) of the EAP and the medium. The heat generated due to dielectric losses in the EAP material can be estimated with:

P=tan δ·f·C·Uₚₚ² (2)

where P is the generated heat (J/s), tan δ is the dielectric dissipation factor (no units), f is the operating frequency (Hz), C is the capacitance (Farad) and Uₚₚ is the peak-to-peak driving voltage (V). In a steady state situation (after an initial heating-up period) the generated heat P will be equal to the transferred heat Q:

P=Q (3)

Substitution of (1) and (2) in (3) leads to the following estimation of the EAP temperature:

$${T}_{eap} = \frac{tan δ f {CU}_{pp}^{2}}{h A} + {T}_{flow} \qquad ( 4 )$$

From equation (4) it appears that the temperature increase (Tₑₐₚ−T_{flow}) scales linearly with the driving frequency. By fitting equation (4) to the measurement in FIG. 25, the heat transfer coefficient in static air in our particular experiment is estimated as h=53 W/m²K when using tan δ=0.1, A=1.5 cm² and C=1 μF The estimated value h=53 W/m²K falls within the range of typical values for heat transfer coefficients in static air, 10-100 W/m²K.

The value h=53 and equation (4) are used to estimate EAP heating at high frequencies and low voltage. FIG. 26 shows the calculated EAP temperature increase Tₑₐₚ−T_{flow} as a function of frequency at low voltage based on the value h=53. FIGS. 25 and 26 show that it should possible to find working points at low and high frequencies (preferred).

Convective heat transfer coefficients for ablation procedures reported in literature cover a wide range, for example 80-3500 W/m²K. These values represent the transfer of heat from tissue to circulating blood.

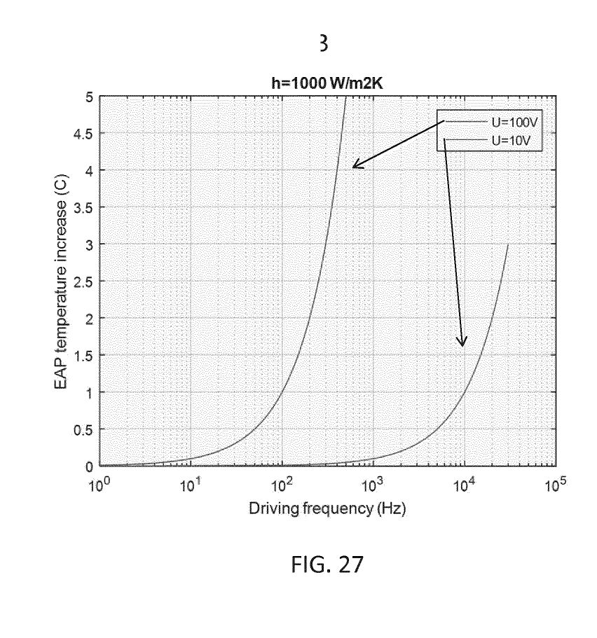

FIG. 27 shows the calculated temperature increase (Tₑₐₚ−T_{flow}) of an actuator, based on an assumed value h=1000 W/m²K (representing operation in blood). The peak-to-peak driving voltage is 100V and 10V respectively. The limit for driving the actuator without damage is for example 1 kHz at 200V in dry conditions. Above this limit the actuator starts to deteriorate rapidly. From initial calculations it can be seen that it is indeed possible to find a working point in blood.

For a (multilayer) electroactive material device, the capacitance is proportional to the area, so according to equation (4), the actuator can be scaled down without influencing Tₑₐₚ−T_{flow} (as a first approximation).

Materials suitable for the EAP layer are known. Electro-active polymers include, but are not limited to, the sub-classes: piezoelectric polymers, electromechanical polymers, relaxor ferroelectric polymers, electrostrictive polymers, dielectric elastomers, liquid crystal elastomers, conjugated polymers, Ionic Polymer Metal Composites, ionic gels and polymer gels.

The sub-class electrostrictive polymers includes, but is not limited to:

Polyvinylidene fluoride (PVDF), Polyvinylidene fluoride-trifluoroethylene (PVDF-TrFE), Polyvinylidene fluoride-trifluoroethylene-chlorofluoroethylene (PVDF-TrFE-CFE), Polyvinylidene fluoride-trifluoroethylene-chlorotrifluoroethylene) (PVDF-TrFE-CTFE), Polyvinylidene fluoride-hexafluoropropylene (PVDF-HFP), polyurethanes or blends thereof.

The sub-class dielectric elastomers includes, but is not limited to:

acrylates, polyurethanes, silicones.

The sub-class conjugated polymers includes, but is not limited to:

polypyrrole, poly-3,4-ethylenedioxythiophene, poly(p-phenylene sulfide), polyanilines.

Ionic devices may be based on ionic polymer-metal composites (IPMCs) or conjugated polymers. An ionic polymer-metal composite (IPMC) is a synthetic composite nanomaterial that displays artificial muscle behavior under an applied voltage or electric field.

In more detail, IPMCs are composed of an ionic polymer like Nafion or Flemion whose surfaces are chemically plated or physically coated with conductors such as platinum or gold, or carbon-based electrodes. Under an applied voltage, ion migration and redistribution due to the imposed voltage across a strip of IPMCs result in a bending deformation. The polymer is a solvent swollen ion-exchange polymer membrane. The field causes cations travel to cathode side together with water. This leads to reorganization of hydrophilic clusters and to polymer expansion. Strain in the cathode area leads to stress in rest of the polymer matrix resulting in bending towards the anode. Reversing the applied voltage inverts the bending.

If the plated electrodes are arranged in a non-symmetric configuration, the imposed voltage can induce all kinds of deformations such as twisting, rolling, torsioning, turning, and non-symmetric bending deformation.

In all of these examples, additional passive layers may be provided for influencing the electrical and/or mechanical behavior of the EAP layer in response to an applied electric field.

The EAP layer of each unit may be sandwiched between electrodes. The electrodes may be stretchable so that they follow the deformation of the EAP material layer. Materials suitable for the electrodes are also known, and may for example be selected from the group consisting of thin metal films, such as gold, copper, or aluminum or organic conductors such as carbon black, carbon nanotubes, graphene, poly-aniline (PANI), poly(3,4-ethylenedioxythiophene) (PEDOT), e.g. poly(3,4-ethylenedioxythiophene) poly(styrenesulfonate) (PEDOT:PSS). Metalized polyester films may also be used, such as metalized polyethylene terephthalate (PET), for example using an aluminum coating.

The invention can be applied in many EAP and photoactive polymer applications, including examples where a passive matrix array of actuators or sensors, or combined sensor and actuators is of interest.

The invention if of interest generally for flow rate sensing, and optionally combined with load sensing, actuation, and temperature sensing for purposes other than for flow rate determination.

In many applications the main function of the product relies on the (local) sensing and optionally also manipulation of human tissue, or the actuation of tissue contacting interfaces. In such applications EAP actuators for example provide unique benefits mainly because of the small form factor, the flexibility and the high energy density. Hence EAP's and photoresponsive polymers can be easily integrated in soft, 3D-shaped and/or miniature products and interfaces. Examples of such applications are:

The invention may be applied to medical and non-medical fields, for example for fluid or gas control components (valves, tubes, pumps) with integrated pressure and flow sensing. In the medical field, it is of interest for intravascular catheters and guidewires and also for respiratory systems.

As discussed above, embodiments make use of a controller. The controller can be implemented in numerous ways, with software and/or hardware, to perform the various functions required. A processor is one example of a controller which employs one or more microprocessors that may be programmed using software (e.g., microcode) to perform the required functions. A controller may however be implemented with or without employing a processor, and also may be implemented as a combination of dedicated hardware to perform some functions and a processor (e.g., one or more programmed microprocessors and associated circuitry) to perform other functions.

Examples of controller components that may be employed in various embodiments of the present disclosure include, but are not limited to, conventional microprocessors, application specific integrated circuits (ASICs), and field-programmable gate arrays (FPGAs).

In various implementations, a processor or controller may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor or controller.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1. A flow sensor comprising:
an electroactive material device arrangement;
a driver circuit,
wherein the driver circuit is arranged to control the electroactive material device arrangement to deliver heat locally to a flowing medium
wherein the flowing medium comprises a flow; and
a controller circuit
wherein the controller circuit is arranged to read sensing signals from the electroactive material device arrangement,
wherein the sensing signals relate to the temperature at the electroactive material device,
wherein the controller circuit is arranged to use the sensing signals to derive a flow measurement,
wherein the controller circuit is arranged to read the sensing signals,
wherein the reading is arranged by performing measurements of an electrical characteristic,
wherein the electrical characteristic comprises an impedance of the electroactive material device or an impedance phase angle of the electroactive material device at a first frequency and at a second frequency,
wherein the second frequency is different from the first frequency,
wherein the controller circuit is arranged to derive a temperature at the electroactive material sensor from the measurements.

2. The sensor as claimed in claim 1,
wherein the electroactive material device has a resonance frequency,
wherein the driver is arranged to provide drive signals at a frequency above the resonance frequency.

3. The sensor as claimed in claim 1, wherein the electroactive material comprises a ferroelectric relaxor polymer.

4. The sensor as claimed in claim 1,
wherein the driver is arranged to deliver heat during a predetermined time period,
wherein the controller circuit is arranged to monitor a subsequent temperature decay function and thereby convert the evolution of the sensing signals over time to a flow measurement.

5. The sensor as claimed in claim 4, wherein the controller circuit is arranged to measure a time period until the temperature reaches a reference temperature, and thereby convert the evolution of the sensing signals over time to a flow measurement.

6. The sensor as claimed in claim 1,
wherein the driver is arranged to deliver heat continuously during a flow sensing time period,
wherein the controller circuit is arranged to read the sensing signals so as to monitor a steady state temperature.

7. The sensor as claimed in claim 1,
wherein the driver is arranged to deliver heat during a flow sensing time period,
wherein the controller circuit is arranged to control the heat delivery rate so as to achieve a predetermined steady state temperature.

8. The sensor as claimed in claim 7, wherein controller circuit is arranged to control a duty cycle.

9. The sensor as claimed in claim 1, wherein the electroactive material device arrangement comprises an arrangement of a first electroactive material device functioning as a heater, a second electroactive material device functioning as a second sensor and third electroactive material device functioning as a third sensor.

10. The sensor as claimed in claim 1, wherein the electroactive material device arrangement also functions as a pressure sensor.

11. The sensor as claimed in claim 1, wherein the controller circuit is arranged to derive an external pressure applied to the electroactive material device arrangement.

12. The sensor as claimed in claim 1,
wherein the first frequency is a resonance frequency at which the electrical characteristic has a maximum or minimum value, an anti-resonance frequency and the second frequency is a frequency at which the electrical characteristic is constant with respect to load.

13. A catheter or guidewire comprising a sensor as claimed in claim 1.

14. A method of measuring a flow rate comprising:
controlling an electroactive material device arrangement to deliver heat locally to a flowing medium, wherein the flowing medium comprises a flow for which the flow rate is to be measured;
reading sensing signals from the electroactive material device arrangement, wherein the sensing signals relate to the temperature at the electroactive material device; and
using the sensing signals to derive a flow measurement,
wherein reading the sensing signals comprises providing sensor readings, deriving from the measurements a temperature at the electroactive material sensor,
wherein the sensor readings are measurements of an electrical characteristic,
wherein the electrical characteristic comprises an impedance of the electroactive material device or an impedance phase angle of the electroactive material device at a first frequency and at a second frequency,
wherein the second frequency is different from the first frequency.

15. The method as claimed in claim 14, further comprising delivering heat by providing drive signals at a frequency above a resonance frequency of the electroactive material device.

16. The method as claimed in claim 14, further comprising delivering heat during a predetermined time period, wherein the controller circuit is arranged to monitor a subsequent temperature decay function and thereby convert the evolution of the sensing signals over time to a flow measurement.

17. The sensor as claimed in claim 1, wherein the electroactive material comprises a PVDF ter-polymer.

18. The sensor as claimed in claim 7, wherein controller circuit is arranged to control a frequency of heat delivery pulses.

19. The sensor as claimed in claim 1, wherein the electroactive material device arrangement also functions as an actuator.

20. The sensor as claimed in claim 1, wherein the controller circuit is arranged to derive a force applied to the electroactive material device arrangement.
